(19) United States
(12) Patent Application Publication
(10) Pub. No.: US 2009/0239146 A1
(43) Pub. Date: Sep. 24, 2009

(54) Non- Aqueous electrolyte secondary battery

(76) Inventors: Hiroshi NAKAGAWA, Saku City (JP); Chihiro Yada, Susono City (JP); Fumiharu Niina, Kobe City (JP); Hiroyuki Fujimoto, Kobe City (JP)

Correspondence Address: KUBOVCIK & KUBOVCIK, SUITE 1105, 1215 SOUTH CLARK STREET, ARLINGTON, VA, 22202, US

(21) Appl. No.: 12/382,497

(22) Filed: Mar. 17, 2009

(30) Foreign Application Priority Data
Mar. 17, 2008 (JP) 2008-67900
Jul. 23, 2008 (JP) 2008-189549
Dec. 16, 2008 (JP) 2008-319939

(51) Int. Cl. H01M 10/26; C07C 59/245

(52) U.S. Cl. 429/207; 562/582

(57) ABSTRACT

A non-aqueous electrolyte secondary battery includes: a positive electrode containing a positive electrode active material, a negative electrode containing a negative electrode active material; and a non-aqueous electrolyte having lithium ion conductivity. The positive electrode includes a lithium-containing transition metal oxide having a layered structure and being represented by the general formula Li₁₊ₓ(NiₐMn_{b}Co_{c})O_{2+α}, where x+a+b+c=1, 0.7≦a+b, 0≦x≦0.1, 0≦c/(a+b)<0.35, 0.7≦a/b≦2.0, and −0.1≦α≦0.1. The non-aqueous electrolyte contains a lithium salt having an oxalato complex as an anion.

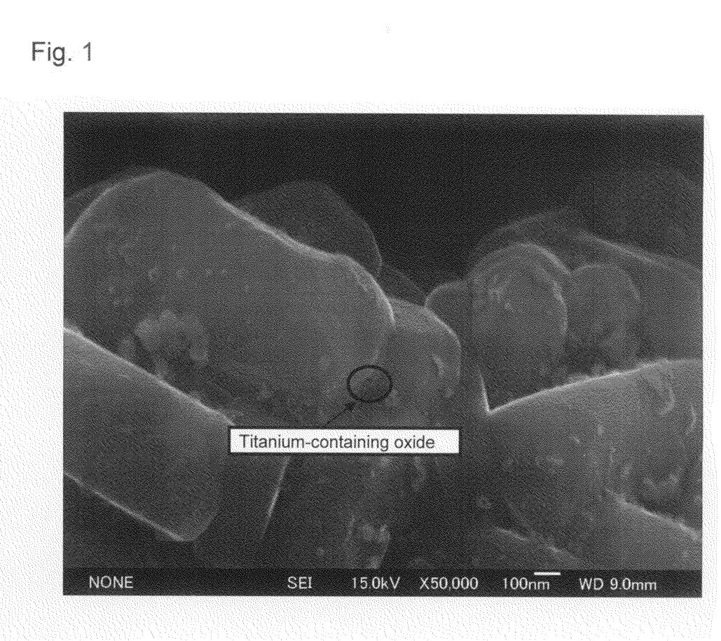

### BACKGROUND OF THE INVENTION

1. Field of the Invention

The present invention relates to non-aqueous electrolyte secondary batteries, such as lithium-ion secondary batteries.

2. Description of Related Art

Lithium-ion secondary batteries have been used widely for the power source of mobile devices, as they are light in weight, small in size, and large in capacity. Recently, they have also drawn attention as a power source for hybrid electric vehicles, and it is expected that they will be used for a wider range of applications.

LiCoO₂ is a common positive electrode active material that is currently used. However, considering the future increase in the uses of Co, a positive electrode active material that does not require Co is needed because Co is an expensive, scarce natural resource.

Potential candidates for such a positive electrode active material are a spinel type LiMn₂O₄ and a layered LiNiO₂. However, a problem with the LiMn₂O₄ is the deterioration at high temperatures, which results from dissolution of Mn. Problems with the LiNiO₂ are that it shows poor thermal stability and is difficult to synthesize and handle.

Recently, a layered lithium-nickel-manganese composite oxide LiNi_{1/2}Mn_{1/2}O₂ has been reported in Japanese Published Unexamined Patent Application No. 2002-428135. Its good capacity and thermal stability have attracted attention. The LiNi_{1/2}Mn_{1/2}O₂, however, has the problem of poor ionic conductivity, leading to poor characteristics, such as high-rate characteristics and input/output power characteristics.

To date, several attempts have been made to improve the discharge characteristics of this material. For example, Japanese Published Unexamined Patent Application No. 2002-110167 discloses a non-aqueous electrolyte secondary battery that employs a lithium-containing transition metal oxide having a layered structure and containing at least nickel and manganese, in which a certain amount of part of the nickel and the manganese is substituted by cobalt. However, since the element substitution by cobalt leads to an increase in the material cost, the effect of cost reduction reduces when the amount of substituting cobalt is large. On the other hand, sufficient input/output power characteristics cannot be obtained when a lithium-containing transition metal composite oxide with a low cobalt substitution amount, specifically, a lithium-containing transition metal composite oxide having a layered structure and being represented by the general formula Li₁₊ₓ(NiₐMn_{b}Co_{c})O_{2+α} (where x+a+b+c=1, 0.7≦a+b, 0<x≦0.1, 0≦c/(a+b)<0.35, 0.7≦a/b≦2.0, and −0.1≦α≦0.1), is used as the positive electrode active material. Accordingly, a technique to improve the input/output power characteristics with such a positive electrode material is needed.

Japanese Published Unexamined Patent Application No. 2006-196250 discloses the use of a lithium salt having an oxalato complex as an anion in order to improve the high-temperature storage performance of a battery that employs a mixture of a lithium-transition metal composite oxide (LiNi_{0.4}Co0.3Mn_{0.3}O₂), which contains Ni and Mn but the content of Co exceeds the range of the foregoing general formula, and a spinel lithium-manganese composite oxide (Li_{1.1}Mn_{1.9}O₄).

Japanese Published Unexamined Patent Application No. 2007-250440 discloses that stable low-temperature performance can be obtained by adding a lithium salt having an oxalato complex as an anion to the electrolyte solution in a battery that has a positive electrode in which the positive electrode active material is a lithium-transition metal composite oxide (LiNi_{0.4}Co_{0.3}Mn_{0.3}O₂), which likewise contains Ni and Mn but the content of Co exceeds the range of the foregoing formula, and the conductive agent of the positive electrode contains fibrous carbon.

Nevertheless, the current state of the art is that it has not been possible to obtain a non-aqueous electrolyte secondary battery that shows good input/output power characteristics using a positive electrode active material composed of a lithium-containing transition metal oxide in which the main components of the transition metals are made of the two elements, nickel and manganese.

### BRIEF SUMMARY OF THE INVENTION

It is an object of the present invention to provide a non-aqueous electrolyte secondary battery that employs a positive electrode active material comprising a lithium-containing transition metal oxide having a layered structure and containing two main transition metal components, nickel and manganese, the non-aqueous electrolyte secondary battery being low in cost and excellent in input/output power characteristics.

In order to accomplish the foregoing and other objects, the present invention provides a non-aqueous electrolyte secondary battery comprising: a positive electrode containing a positive electrode active material, the positive electrode active material comprising a lithium-containing transition metal oxide having a layered structure and being represented by the general formula Li₁₊ₓ(NiₐMn_{b}Co_{c})O_{2+α}, where x+a+b+c=1, 0.7≦a+b, 0<x≦0.1, 0≦c/(a+b)<0.35, 0.7≦a/b≦2.0, and −0.1≦α≦0.1; a negative electrode containing a negative electrode active material; and a non-aqueous electrolyte having lithium ion conductivity and containing a lithium salt having an oxalato complex as an anion.

In the present invention, the positive electrode active material may be a lithium-containing transition metal composite oxide in which a titanium-containing oxide is adhered to the surface of the lithium-containing transition metal composite oxide.

The present invention makes it possible to obtain a non-aqueous electrolyte secondary battery that employs a positive electrode active material comprising a lithium-containing transition metal oxide having a layered structure and containing two main transition metal components, nickel and manganese, the non-aqueous electrolyte secondary battery being low in cost and excellent in input/output power characteristics.

### BRIEF DESCRIPTION OF THE DRAWING

The drawing is a scanning electron microscope (SEM) photograph showing the positive electrode active material used in Example 6 according to the present invention.

### DETAILED DESCRIPTION OF THE INVENTION

A non-aqueous electrolyte secondary battery of the present invention comprises: a positive electrode containing a positive electrode active material, a negative electrode containing a negative electrode active material; and a non-aqueous electrolyte having lithium ion conductivity. The positive electrode comprises a lithium-containing transition metal oxide having a layered structure and being represented by the general formula Li₁₊ₓ(NiₐMn_{b}Co_{c})O_{2+α}, where x+a+b+c=1, 0.7≦a+b, 0<x≦0.1, 0≦c/(a+b)<0.35, 0.7≦a/b≦2.0, and −0.1≦α≦0.1. The non-aqueous electrolyte contains a lithium salt having an oxalato complex as an anion.

In addition, the present invention may employ a positive electrode active material in which a titanium-containing oxide is adhered to the surface of the lithium-containing transition metal composite oxide.

The present invention employs a positive electrode active material comprising a lithium-containing transition metal composite oxide containing Ni and Mn as the main components, having a low Co content, and being represented by the foregoing general formula, or a lithium-containing transition metal composite oxide in which a titanium-containing oxide is adhered to the surface of the foregoing lithium-containing transition metal composite oxide. In addition, in the present invention, the non-aqueous electrolyte contains a lithium salt having an oxalato complex as an anion. Therefore, it is possible to obtain a non-aqueous electrolyte secondary battery that is excellent in input/output power characteristics and low in cost.

The details of the effect of the lithium salt having an oxalato complex as an anion are not yet clear. However, it is believed that the anion of the oxalato complex decomposes during the initial charge and a surface film forms on the positive electrode active material surface, reducing the reaction resistance of the insertion and deinsertion of the lithium ions at the surface of the positive electrode active material. Thereby, it is believed that the I-V resistance is reduced, and good input/output power characteristics can be obtained.

The lithium-containing transition metal oxide used in the present invention has a layered structure, contains nickel and manganese as the main components of the transition metals, and is represented by the general formula Li₁₊ₓ(NiₐMn_{b}Co_{c})O_{2+α} (where x+a+b+c=1, 0.7≦a+b, 0<x≦0.1, 0≦c/(a+b)<0.35, 0.7≦a/b≦2.0, −0.1≦α≦0.1). In the general formula, x+a+b+c=1, which means that Li in excess of 1 is in the transition metal sites. The ratio a/b, which is the composition ratio of nickel and manganese, is in the range 0.7≦a/b≦2.0. When the ratio a/b exceeds 2.0, the proportion of Ni is large, degrading the thermal stability, as shown in the later-described reference experiments. On the other hand, when the ratio a/b is less than 0.7, the proportion of Mn is large, forming an impurity phase and lowering the capacity. Considering the balance between thermal stability and capacity, it is more preferable that the ratio a/b be in the range 0.9≦a/b≦1.1.

The variable x, which shows the amount of Li in excess of 1, is in the range 0<x≦0.1. The input/output power characteristics can be enhanced when 0<x. However, when x>0.1, the amount of remaining alkali on the active material surface becomes large, causing gelation of the slurry during the fabrication process of the battery. Moreover, the amounts of the transition metals that undergo the oxidation-reduction reactions reduce, degrading the capacity. It is more preferable that x be in the range 0.05≦x≦0.1.

In addition, the variables a and b should satisfy the expression 0.7≦a+b. If a+b is less than 0.7, the content of nickel and manganese is low, and the cobalt content is large. Therefore, it is impossible to obtain a low cost non-aqueous electrolyte secondary battery.

In the foregoing general formula, the variables a, b, and c should satisfy the expression 0≦c/(a+b)<0.35. If c/(a+b) is equal to or greater than 0.35, the content of nickel and manganese is low, and the content of cobalt is high. Therefore, it is impossible to obtain a low cost non-aqueous electrolyte secondary battery.

In particular, it is preferable that c be 0 in the foregoing general formula. In other words, it is preferable that the lithium-containing transition metal composite oxide does not contain Co. Accordingly, it becomes unnecessary to use Co, which is an expensive, scarce natural resource, and moreover, the effect of reducing the I-V resistance becomes greater. In addition, it is preferable that in the foregoing general formula, c be 0 and at the same time, a=b. This enables a further greater effect of reducing the I-V resistance to be obtained.

In the foregoing general formula, α, which shows the amount of oxygen deficiency or oxygen excess, is in the range −0.1≦α≦0.1. The lithium-containing transition metal composite oxide in the present invention can obtain sufficient advantageous effects of the present invention even if it has oxygen deficiency or oxygen excess. Nevertheless, when α is outside the above-described range, the crystal structure may be impaired and the advantageous effects of the present invention may not be obtained sufficiently because of oxygen deficiency or oxygen excess.

It is preferable that the lithium-containing transition metal composite oxide in the present invention have a secondary particle size of from 5 μm to 15 μm. It is also preferable that the lithium-containing transition metal composite oxide have a primary particle size of from 0.5 μm to 2 μm. If the secondary particle size and the primary particle size are above the foregoing range, the discharge performance may degrade. On the other hand, if they are below the foregoing range, the reactivity of the active material with the non-aqueous electrolyte may increase, degrading the storage performance and the like.

In addition, as described above, the present invention may employ a positive electrode active material in which a titanium-containing oxide is adhered to the surface of the lithium-containing transition metal composite oxide. When the titanium-containing oxide is adhered to the surface, the reaction resistance in the insertion and deinsertion of lithium to/from the lithium-containing transition metal oxide can be reduced. As a result, the input/output power characteristics can be improved further. It is preferable that the content of the titanium-containing oxide in the positive electrode active material be from 0.05 weight % to 1.0 weight %, based on the content of titanium, and more preferably from 0.05 weight % to 0.5 weight %. If the content of the titanium-containing oxide is less than 0.05 weight %, the effect originating from the adherence of the titanium-containing oxide may not be sufficient. On the other hand, if the content of the titanium-containing oxide exceeds 1.0 weight %, the characteristics may degrade.

Although the type of the titanium-containing oxide to be adhered to the surface of the lithium-containing transition metal oxide is not particularly limited, it is preferable that it be a lithium-titanium oxide or a titanium oxide. Examples include compounds such as Li₂TiO₃, Li₄Ti₅O₁₂, and TiO₂, and mixtures thereof.

The method for adhering the titanium-containing oxide to the lithium-containing transition metal oxide surface is not particularly limited. For example, predetermined amounts of a lithium-containing transition metal oxide and a titanium-containing oxide may be mixed together using a mechanofusion system or the like, to adhere the titanium-containing oxide to the lithium-containing transition metal oxide surface. In this case, it is preferable that a heat treatment be carried out after the titanium-containing oxide is adhered. By conducting the heat treatment, the titanium-containing oxide is allowed to adhere to the lithium-containing transition metal oxide surface more firmly. It is preferable that the sintering temperature in this process be lower than the decomposition temperature of the lithium-containing transition metal oxide, more preferably in the range of from 300° C. to 900° C.

An example of the titanium-containing oxide to be mixed with the lithium-containing transition metal oxide includes titanium oxide (TiO₂). Preferable examples of the titanium oxide include those having an average particle size of 30 nm to 500 nm.

In the present invention, the positive electrode preferably further contains a lithium-manganese composite oxide having a spinel structure, with which the above-described lithium-transition metal composite oxide may be mixed when used as the positive electrode active material. In this case, the input/output power characteristics of can be improved further.

The lithium-manganese composite oxide having a spinel structure may contain one or a plurality of elements selected from the group consisting of B, F, Mg, Al, Ti, Cr, V, Fe, Co, Ni, Cu, Zn, Nb, and Zr. Among them, it is particularly preferable that at least one of Mg and Al be contained. When at least one of Mg and Al is contained, it is possible to achieve higher cycle performance and better high-temperature storage performance.

In the present invention, a preferable lithium-manganese composite oxide having a spinel structure is represented by the general formula Li_{1+y}Mn_{d}AₑO_{4+β}, where A is at least one of Mg and Al, y+d+e=2, 0<e, 0<y+e<0.3, and −0.1≦β≦0.1.

It is preferable that the weight ratio of the lithium-containing transition metal composite oxide and the lithium-manganese composite oxide having a spinel structure in the positive electrode active material (the lithium-containing transition metal composite oxide:the lithium-manganese composite oxide having a spinel structure) be from about 90:10 to about 30:70, more preferably from about 70:30 to about 50:50.

It should be noted that when the lithium-manganese composite oxide having a spinel structure is used alone as the positive electrode active material, the input/output power characteristics do not improve sufficiently even with the use of the electrolyte solution containing an oxalato complex salt.

In the present invention, it is preferable that the lithium salt having an oxalato complex as an anion (hereinafter referred to as an “oxalato complex salt”) be contained in the non-aqueous electrolyte at a concentration of from 0.05 mole /liter to 0.3 mole /liter. If the concentration is less than 0.05 mole/liter, the effect of improving the input/output power characteristics may not be sufficient. On the other hand, if the concentration exceeds 0.3 mole/liter, the rated discharge capacity of the battery may decrease considerably. A more preferable range of the concentration of the oxalato complex salt in the non-aqueous electrolyte is from 0.1 mole/liter to 0.2 mole/liter. More desirable input/output power characteristics can be obtained within this range.

In addition, the oxalato complex salt in the present invention may be a lithium salt having an anion in which C₂O₄²⁻ coordinates to the central atom. It is possible to use a substance represented as Li[M(C₂O₄)ₓR_{y}], where M is an element selected from the group consisting of transition metals and Group IIIb (13), Group IVb (14), and Group Vb (15) elements of the periodic table, R is a group selected from halogens, alkyl groups, halogen substituted alkyl groups, x is a positive integer, and y is 0 or a positive integer. Specific examples include Li[B(C₂O₄)₂], Li[B(C₂O₄)F₂], Li[P(C₂O₄)F₄], and Li[P(C₂O₄)₂F₂]. In particular, it is desirable to use lithium bis(oxalato)borate (Li[B(C₂O₄)₂]), in order to form a stable surface film on the negative electrode surface even under a high temperature environment.

The negative electrode active material used in the present invention may be any material including carbon, alloys, and metal oxides, as long as it can reversibly intercalate and deintercalate lithium. From the viewpoint of material cost, it is preferable to use a carbon material. Examples of the carbon material include natural graphite, artificial graphite, mesophase pitch-based carbon fiber (MCF), mesocarbon microbead (MCMB), coke, hard carbon, fullerenes, and carbon nanotubes. Among these materials, it is particularly preferable to use an amorphous carbon-coated graphite, in which a graphite material is coated with amorphous carbon from the viewpoint of improving the charge-discharge characteristics.

The lithium salt of the non-aqueous electrolyte used in the present invention may be any lithium salt that is conventionally used as an electrolyte for non-aqueous electrolyte secondary batteries. It is preferable that such a lithium salt contain at least one element selected from the group consisting of P, B, F, O, S, N, and Cl. Specific usable examples include LiPF₆, LiBF₄, LiCF₃SO₃, LiN(CF₃SO₂)₂, LiN(C₂F₅SO₂)₂, LiN(CF₃SO₂)(C₄F₉SO₂), LiC(C₂F₅SO₂)₃, LiAsF₆, LiClO₄, and mixtures thereof. It is particularly preferable to use LiPF₆, in order to obtain both good input/output power characteristics and high durability of the battery.

The solvent of the non-aqueous electrolyte used in the present invention may be any solvent that has conventionally been used as a solvent for an electrolyte in non-aqueous electrolyte secondary batteries. Examples of the solvent include: cyclic carbonates, such as ethylene carbonate, propylene carbonate, butylene carbonate, and vinylene carbonate; and chain carbonates, such as dimethyl carbonate, methyl ethyl carbonate, and diethyl carbonate. In particular, it is preferable that the solvent be a mixed solvent of a cyclic carbonate and a chain carbonate, which have a low viscosity, a low melting point, and high lithium ion conductivity. In the just-mentioned mixed solvent, it is preferable that the volume ratio of the cyclic carbonate and the chain carbonate be within the range of from 2/8 to 5/5. It is also possible to use an ionic liquid as the solvent for the electrolyte. In this case, the cationic species and the anionic species are not particularly limited; however, it is preferable to use a combination in which the cation is pyridinium cation, imidazolium cation, and quaternary ammonium cation, and the anion is fluorine-containing imide-based anion, from the viewpoints of obtaining low viscosity, electrochemical stability, and hydrophobicity.

It is also possible to add a film-forming agent, such as vinylene carbonate, vinyl ethylene carbonate, ethylene sulfite, and fluoroethylene carbonate, to the solvent of the non-aqueous electrolyte. In particular, it is preferable that vinylene carbonate be contained in order to obtain a stable surface film even after the charge-discharge cycles have been repeated.

Hereinbelow, the present invention is described in further detail. It should be construed, however, that the present invention is not limited to the following preferred embodiments but various changes and modifications are possible without departing from the scope of the invention.

### EXAMPLE 1

### Preparation of Positive Electrode

A lithium-containing transition metal composite oxide was prepared as a positive electrode active material in the following manner. Ni_{0.5}Mn_{0.5}(OH)₂ and Li₂CO₃ were mixed, and the resulting mixture was sintered in an air atmosphere at 900° C. for 20 hours. Thus, a lithium-containing transition metal composite oxide was prepared. The obtained lithium-containing transition metal composite oxide was observed by ICP spectrometry. As a result, it was found that the composition of the lithium-containing transition metal composite oxide obtained was Li_{1.06}Ni_{0.47}MN_{0.47}o₂.

The obtained lithium-containing transition metal composite oxide had an average secondary particle size of 6 μm and a specific surface area of 0.6 m²/g. It was confirmed by X-ray diffraction analysis that the obtained lithium-containing transition metal composite oxide had a crystal structure belonging to the space group R3m.

The lithium-containing transition metal composite oxide prepared in the above-described manner, a graphite material as a conductive agent, and a N-methyl-2-pyrrolidone solution in which polyvinylidene fluoride was dissolved, as a binder agent, were mixed so that the weight ratio of the active material, the conductive agent, and the binder agent became 92:5:3, to prepare a positive electrode slurry. The slurry thus prepared was applied onto an aluminum foil serving as a current collector and then dried. Thereafter, the aluminum foil coated with the positive electrode slurry was compressed with rollers, and a current collector tab was attached thereto. Thus, a positive electrode was prepared.

### Preparation of Negative Electrode

Graphite in which the surface was coated with amorphous carbon, serving as a negative electrode active material, a water dispersion of styrene-butadiene rubber (SBR), serving a binder agent, and an aqueous solution in which carboxymethylcellulose (CMC) was dissolved, serving as a thickening agent, were kneaded so that the weight ratio of the active material, the binder agent, and the thickening agent became 98.9:0.4:0.7, to prepare a negative electrode slurry. The slurry thus prepared was applied onto a copper foil serving as a current collector, and then dried. Thereafter, the resultant material was compressed with rollers, and a current collector tab was attached thereto. Thus, a negative electrode was prepared.

### Preparation of Electrolyte Solution

LiPF₆ as a solute was dissolved at a concentration of 1M (mole/liter) in a solvent in which ethylene carbonate (EC), methyl ethyl carbonate (MEC), and diethyl carbonate (DEC) were mixed at a volume ratio of 3:4:3. Then, 1%, by weight ratio, of vinylene carbonate (VC) was added to the resultant electrolyte solution. Thereafter, lithium bis(oxalato)borate (LiBOB) was further added thereto at a concentration of 0.1M, to thus prepare an electrolyte solution.

### Fabrication of Non-Aqueous Electrolyte Secondary Battery

The positive electrode and the negative electrode prepared in the above-described manner were coiled with a polyethylene separator interposed therebetween, to prepare a wound electrode assembly. In a glove box under an argon atmosphere, the wound electrode assembly was enclosed in a battery can together with the electrolyte solution. Thus, a cylindrical 18650 size non-aqueous electrolyte secondary battery A1 was fabricated.

The fabricated battery was charged at a constant current of 1000 mA to 4.2 V and further charged at a constant voltage of 4.2 V to a current of 50 mA. Then, the battery was discharged at 300 mA to 2.4 V. The capacity obtained in this process was defined as the battery discharge capacity.

### Measurement of I-V Profile

Samples of the non-aqueous electrolyte secondary battery fabricated in the above-described manner were charged at a charge current of 200 mA to a state of charge (SOC) of 50% at a temperature of 25° C., and they were charged and discharged for 10 seconds at respective currents of 0.1 A, 0.5 A, 1 A, and 2 A. The battery voltages were measured respectively, and the current values and the battery voltages were plotted to obtain the I-V profile during charge and discharge. From the gradient of the obtained straight line, the charge side I-V resistance (mΩ) and the discharge side I-V resistance (mΩ) were obtained.

### EXAMPLE 2

A non-aqueous electrolyte secondary battery A2 was fabricated and the I-V profile thereof was measured in the same manner as described in Example 1, except that LiBOB was dissolved at a concentration of 0.05M when preparing the electrolyte solution.

### EXAMPLE 3

A non-aqueous electrolyte secondary battery A3 was fabricated and the I-V characteristics were measured in the same manner as described in Example 1, except that LiBOB was dissolved at a concentration of 0.15M when preparing the electrolyte solution.

### EXAMPLE 4

A non-aqueous electrolyte secondary battery A4 was fabricated and the I-V characteristics were measured in the same manner as described in Example 1, except that LiBOB was dissolved at a concentration of 0.2M when preparing the electrolyte solution.

### EXAMPLE 5

A non-aqueous electrolyte secondary battery A5 was fabricated and the I-V characteristics were measured in the same manner as described in Example 1, except that LiBOB was dissolved at a concentration of 0.3M when preparing the electrolyte solution.

### EXAMPLE 6

A predetermined amount of TiO₂ having an average particle size of 50 nm was weighed and mixed with the lithium-containing transition metal composite oxide Li_{1.06}Ni_{0.47}Mn0.47O₂ prepared in Example 1. Thereafter, the mixture was sintered at 700° C. in the air in order to cause the titanium-containing oxide to adhere to the Li_{1.06}Ni_{0.47}Mn_{0.47}O₂ surface more firmly, and the resultant substance was used as the positive electrode active material. The content of the titanium in the positive electrode active material prepared in this manner was 0.24 weight %. A non-aqueous electrolyte secondary battery A6 of Example 6 was fabricated and the I-V characteristics were measured in the same manner as described in Example 1, except that the lithium-containing transition metal composite oxide obtained in the just-described manner, in which the titanium-containing oxide was adhered to the surface thereof, was used as the positive electrode active material.

It should be noted that the drawing shows a SEM photograph of the positive electrode active material used in Example 6. It was observed that microparticles having an average particle size of 50 nm were dispersed and adhered substantially uniformly over the surface of the Li_{1.06}Ni_{0.47}Mn_{0.47}O₂. Here, it is believed that the microparticles adhering on the surface were composed of a source material TiO₂, a lithium-titanium oxide (Li—Ti—O) such as Li₂TiO₃ or Li₄Ti₅O₁₂, which was produced by the reaction between the TiO₂ and the remaining lithium on the Li1.06Ni_{0.47}Mn_{0.47}O₂ surface, or a mixture thereof.

### EXAMPLE 7

In the present example, except for using Ni_{0.6}Mn_{0.4}(OH)₂, a lithium-containing transition metal composite oxide as the positive electrode active material was prepared in the same manner as described in Example 1 above, and using the resultant positive electrode active material, a positive electrode was prepared. The composition of the obtained lithium-containing transition metal composite oxide was found to be Li_{1.07}Ni_{0.56}Mn_{0.37}O₂ by ICP spectrometry.

The obtained lithium-containing transition metal composite oxide had an average particle size of 6 μm and a specific surface area of 0.5 m²/g. It was confirmed by X-ray diffraction analysis that the obtained lithium-containing transition metal composite oxide had a crystal structure belonging to the space group R3m.

Except for using the positive electrode prepared in the present example, a non-aqueous electrolyte secondary battery A7 was fabricated and the I-V characteristics were measured in the same manner as described in Example 1.

### EXAMPLE 8

In the present example, a lithium-containing transition metal composite oxide as the positive electrode active material was prepared in the same manner as described in Example 1 above, except for using Ni_{0.45}Co_{0.1}Mn_{0.45}(OH)₂, to prepare a positive electrode. The composition of the obtained lithium-containing transition metal composite oxide was found to be Li1.07Ni0.42Co_{0.09}Mn_{0.42}O₂ by ICP spectrometry. Therefore, the value of c/(a+b) in the foregoing general formula was 0.11 in the present example.

The obtained lithium-containing transition metal composite oxide had an average particle size of 7 μm and a specific surface area of 0.6 m²/g. It was confirmed by X-ray diffraction analysis that the obtained lithium-containing transition metal composite oxide had a crystal structure belonging to the space group R3m.

Except for using the positive electrode prepared in the present example, a non-aqueous electrolyte secondary battery A8 was fabricated and the I-V characteristics were measured in the same manner as described in Example 1.

### EXAMPLE 9

In the present example, a non-aqueous electrolyte secondary battery A9 was fabricated and the I-V characteristics were measured in the same manner as described in Example 1 above, except that the positive electrode active material was a 5:5 weight ratio mixture of Li_{1.07}Ni_{0.42}Co_{0.09}Mn_{0.42}O₂ and a spinel lithium-manganese composite oxide Li1.06Mn_{1.89}Mg_{0.05}O₄ (i.e., Li_{1.07}Ni_{0.42}Co_{0.09}Mn_{0.42}O₂:Li_{1.06}Mn_{1.89}Mg_{0.05}O₄=5:5).

### EXAMPLE 10

In the present example, a non-aqueous electrolyte secondary battery A10 was fabricated and the I-V characteristics were measured in the same manner as described in Example 9 above, except that the positive electrode active material was a 7:3 weight ratio mixture of Li1.07Ni_{0.42}Co_{0.09}Mn_{0.42}O₂ and a spinel lithium-manganese composite oxide Li_{1.06}Mn_{1.89}Mg_{0.5}O₄ (i.e., Li_{1.07}Ni_{0.42}Co_{0.09}Mn_{0.42}O₂:Li_{1.06}Mn_{1.89}Mg_{0.05}O₄=7:3).

### EXAMPLE 11

In the present example, a non-aqueous electrolyte secondary battery A11 was fabricated and the I-V characteristics were measured in the same manner as described in Example 1 above, except that the positive electrode active material was a 5:5 weight ratio mixture of Li_{1.07}Ni_{0.56}Mn_{0.37}O₂ and a spinel lithium-manganese composite oxide Li_{1.06}Mn_{1.89}Mg_{0.05}O₄ (i.e., Li_{1.07}Ni_{0.56}Mn_{0.37}O₂:Li_{1.06}Mn_{1.89}Mg_{0.05}O₄=5:5).

### COMPARATIVE EXAMPLE 1

A non-aqueous electrolyte secondary battery B1 was fabricated and the I-V characteristics were measured in the same manner as described in Example 1, except that no LiBOB was contained when preparing the electrolyte solution.

### COMPARATIVE EXAMPLE 2

### Preparation of Positive Electrode

A lithium-containing transition metal composite oxide was prepared as a positive electrode active material in the following manner. Ni_{0.4}Co_{0.3}Mn_{0.3}(OH)₂ and Li₂CO₃ were mixed, and the resulting mixture was sintered in an air atmosphere at 900° C. for 20 hours. Thus, a lithium-containing transition metal composite oxide was prepared. The composition of the obtained lithium-containing transition metal composite oxide was found to be Li_{1.07}Ni_{0.37}Co_{0.28}Mn_{0.28}O₂ by ICP spectrometry. Therefore, the value of c/a+b in the foregoing general formula was 0.43 for this composition.

The obtained lithium-containing transition metal composite oxide had an average particle size of 13 μm and a specific surface area of 0.3 m²/g. It was confirmed by X-ray diffraction analysis that the obtained lithium-containing transition metal composite oxide had a crystal structure belonging to the space group R3m.

The lithium-containing transition metal composite oxide prepared in the above-described manner, a graphite material as a conductive agent, and a N-methyl-2-pyrrolidone solution in which polyvinylidene fluoride was dissolved, as a binder agent, were mixed so that the weight ratio of the active material, the conductive agent, and the binder agent became 92:5:3, to prepare a positive electrode slurry. The slurry thus prepared was applied onto an aluminum foil serving as a current collector and then dried. Thereafter, the aluminum foil coated with the positive electrode slurry was compressed with rollers, and a current collector tab was attached thereto. Thus, a positive electrode was prepared.

Except for using the positive electrode prepared in this manner, a non-aqueous electrolyte secondary battery B2 was fabricated and the I-V characteristics were measured in the same manner as described in Example 1.

### COMPARATIVE EXAMPLE 3

A non-aqueous electrolyte secondary battery B3 was fabricated and the I-V characteristics were measured in the same manner as described in Comparative Example 2, except that no LiBOB was contained when preparing the electrolyte solution.

### COMPARATIVE EXAMPLE 4

A non-aqueous electrolyte secondary battery B4 was fabricated and the I-V characteristics were measured in the same manner as described in Example 6, except that no LiBOB was contained when preparing the electrolyte solution.

### COMPARATIVE EXAMPLE 5

A non-aqueous electrolyte secondary battery B5 was fabricated and the I-V characteristics were measured in the same manner as described in Example 7, except that no LiBOB was contained when preparing the electrolyte solution.

### COMPARATIVE EXAMPLE 6

A non-aqueous electrolyte secondary battery B6 was fabricated and the I-V characteristics were measured in the same manner as described in Example 8, except that no LiBOB was contained when preparing the electrolyte solution.

### COMPARATIVE EXAMPLE 7

In the present example, except for using Ni_{0.35}Co_{0.35}Mn_{0.3}(OH)₂, a lithium-containing transition metal composite oxide as the positive electrode active material was prepared in the same manner as described in Example 1 above, and using the resultant positive electrode active material, a positive electrode was prepared. The composition of the obtained lithium-containing transition metal composite oxide was found to be Li_{1.07}Ni_{0.33}Co_{0.33}Mn_{0.28}O₂ by ICP spectrometry. Therefore, the value of c/(a+b) in the foregoing general formula was 0.54 in the present comparative example.

The obtained lithium-containing transition metal composite oxide had an average particle size of 12 μm and a specific surface area of 0.2 m²/g. It was confirmed by X-ray diffraction analysis that the obtained lithium-containing transition metal composite oxide had a crystal structure belonging to the space group R3m.

Except for using the positive electrode prepared in this comparative example, a non-aqueous electrolyte secondary battery B7 was fabricated and the I-V characteristics were measured in the same manner as described in Example 1.

### COMPARATIVE EXAMPLE 8

A non-aqueous electrolyte secondary battery B8 was fabricated and the I-V characteristics were measured in the same manner as described in Comparative Example 7, except that no LiBOB was contained when preparing the electrolyte solution.

### COMPARATIVE EXAMPLE 9

A non-aqueous electrolyte secondary battery B9 was fabricated and the I-V characteristics were measured in the same manner as described in Example 1 above, except that the lithium-manganese composite oxide having a spinel structure Li_{1.06}Mn_{1.89}Mg_{0.05}O₄ alone was used as the positive electrode active material when preparing the positive electrode.

### COMPARATIVE EXAMPLE 10

A non-aqueous electrolyte secondary battery B10 was fabricated and the I-V characteristics were measured in the same manner as described in Comparative Example 9, except that no LiBOB was contained when preparing the electrolyte solution.

### COMPARATIVE EXAMPLE 11

A non-aqueous electrolyte secondary battery B11 was fabricated and the I-V characteristics were measured in the same manner as described in Example 9, except that no LiBOB was contained when preparing the electrolyte solution.

### COMPARATIVE EXAMPLE 12

A non-aqueous electrolyte secondary battery B12 was fabricated and the I-V characteristics were measured in the same manner as described in Example 10, except that no LiBOB was contained when preparing the electrolyte solution.

### COMPARATIVE EXAMPLE 13

In the present example, a non-aqueous electrolyte secondary battery B13 was fabricated and the I-V characteristics were measured in the same manner as described in Example 1 above, except that the positive electrode active material was a 5:5 weight ratio mixture of a lithium-containing transition metal composite oxide Li_{1.07}Ni_{0.33}Co_{0.33}Mn_{0.28}O₂ and a spinel lithium-manganese composite oxide Li_{1.06}Mn_{1.89}Mg_{0.05}O₄ (i.e., Li_{1.07}Ni_{0.33}Co_{0.33}Mn_{0.28}O₂:Li_{1.06}Mn_{1.89}Mg_{0.05}O₄=5:5) when preparing the positive electrode.

### COMPARATIVE EXAMPLE 14

A non-aqueous electrolyte secondary battery B14 was fabricated and the I-V characteristics were measured in the same manner as described in Comparative Example 13, except that no LiBOB was contained when preparing the electrolyte solution.

### COMPARATIVE EXAMPLE 15

A non-aqueous electrolyte secondary battery B15 was fabricated and the I-V characteristics were measured in the same manner as described in Example 11, except that no LiBOB was contained when preparing the electrolyte solution.

The evaluation results of the discharge capacity and I-V profile of the 18650 batteries measured in the foregoing manners are shown in Table 1 below.

TABLE 1

| | | | | Charge | Discharge |
|---|---|---|---|---|---|
| | | LiBOB | Battery | side | side |
| | Positive electrode active material | concentration | discharge | I-V | I-V |
| | composition | in electrolyte | capacity | resistance | resistance |
| Ex. 1 | Li_{1.06}Ni_{0.47}Mn_{0.47}O₂ | 0.1M | 817 mAh | 58 mΩ | 64 mΩ |
| Ex. 2 | Li_{1.06}Ni_{0.47}Mn_{0.47}O₂ | 0.05M | 841 mAh | 63 mΩ | 70 mΩ |
| Ex. 3 | Li_{1.06}Ni_{0.47}Mn_{0.47}O₂ | 0.15M | 786 mAh | 60 mΩ | 65 mΩ |
| Ex. 4 | Li_{1.06}Ni_{0.47}Mn_{0.47}O₂ | 0.2M | 767 mAh | 60 mΩ | 64 mΩ |
| Ex. 5 | Li_{1.06}Ni_{0.47}Mn_{0.47}O₂ | 0.3M | 741 mAh | 63 mΩ | 66 mΩ |
| Ex. 6 | Titanium oxide-containing | 0.1M | 811 mAh | 55 mΩ | 63 mΩ |
| | Li_{1.06}Ni_{0.47}Mn_{0.47}O₂ | | | | |
| Ex. 7 | Li_{1.07}Ni_{0.56}Mn_{0.37}O₂ | 0.1M | 894 mAh | 50 mΩ | 54 mΩ |
| Ex. 8 | Li_{1.07}Ni_{0.42}Co_{0.09}Mn_{0.42}O₂ | 0.1M | 825 mAh | 39 mΩ | 41 mΩ |
| Ex. 9 | Li_{1.07}Ni_{0.42}Co_{0.09}Mn_{0.42}O₂ (50%) + | 0.1M | 1008 mAh | 33 mΩ | 33 mΩ |
| | Li_{1.06}Mn_{1.89}Mg_{0.05}O₄ (50%) | | | | |
| Ex. 10 | Li_{1.07}Ni_{0.42}Co_{0.09}Mn_{0.42}O₂ (70%) + | 0.1M | 827 mAh | 37 mΩ | 37 mΩ |
| | Li_{1.06}Mn_{1.89}Mg_{0.05}O₄ (30%) | | | | |
| Ex. 11 | Li_{1.07}Ni_{0.56}Mn_{0.37}O₂ (50%) + | 0.1M | 852 mAh | 36 mΩ | 36 mΩ |
| | Li_{1.06}Mn_{1.89}Mg_{0.05}O₄ (50%) | | | | |
| Comp. | Li_{1.06}Ni_{0.47}Mn_{0.47}O₂ | No additive | 848 mAh | 73 mΩ | 85 mΩ |
| Ex. 1 | | | | | |
| Comp. | Li_{1.07}Ni_{0.37}Co_{0.28}Mn_{0.28}O₂ | 0.1M | 853 mAh | 33 mΩ | 32 mΩ |
| Ex. 2 | | | | | |
| Comp. | Li_{1.07}Ni_{0.37}Co_{0.28}Mn_{0.28}O₂ | No additive | 889 mAh | 34 mΩ | 33 mΩ |
| Ex. 3 | | | | | |
| Comp. | Titanium oxide-containing | No additive | 848 mAh | 58 mΩ | 71 mΩ |
| Ex. 4 | Li_{1.06}Ni_{0.47}Mn_{0.47}O₂ | | | | |
| Comp. | Li_{1.07}Ni_{0.56}Mn_{0.37}O₂ | No additive | 922 mAh | 57 mΩ | 64 mΩ |
| Ex. 5 | | | | | |
| Comp. | Li_{1.07}Ni_{0.42}Co_{0.09}Mn_{0.42}O₂ | No additive | 874 mAh | 43 mΩ | 47 mΩ |
| Ex. 6 | | | | | |
| Comp. | Li_{1.07}Ni_{0.33}Co_{0.33}Mn_{0.28}O₂ | 0.1M | 951 mAh | 32 mΩ | 32 mΩ |
| Ex. 7 | | | | | |
| Comp. | Li_{1.07}Ni_{0.33}Co_{0.33}Mn_{0.28}O₂ | No additive | 973 mAh | 32 mΩ | 33 mΩ |
| Ex. 8 | | | | | |
| Comp. | Li_{1.06}Mn_{1.89}Mg_{0.05}O₄ | 0.1M | 773 mAh | 34 mΩ | 35 mΩ |
| Ex. 9 | | | | | |
| Comp. | Li_{1.06}Mn_{1.89}Mg_{0.05}O₄ | No additive | 792 mAh | 33 mΩ | 34 mΩ |
| Ex. 10 | | | | | |
| Comp. | Li_{1.07}Ni_{0.42}Co_{0.09}Mn_{0.42}O₂ (50%) + | No additive | 1023 mAh | 40 mΩ | 41 mΩ |
| Ex. 11 | Li_{1.06}Mn_{1.89}Mg_{0.05}O₄ (50%) | | | | |
| Comp. | Li_{1.07}Ni_{0.42}Co_{0.09}Mn_{0.42}O₂ (70%) + | No additive | 867 mAh | 39 mΩ | 39 mΩ |
| Ex. 12 | Li_{1.06}Mn_{1.89}Mg_{0.05}O₄ (30%) | | | | |
| Comp. | Li_{1.07}Ni_{0.33}Co_{0.33}Mn_{0.28}O₂ (50%) + | 0.1M | 783 mAh | 31 mΩ | 31 mΩ |
| Ex. 13 | Li_{1.06}Mn_{1.89}Mg_{0.05}O₄ (50%) | | | | |
| Comp. | Li_{1.07}Ni_{0.33}Co_{0.33}Mn_{0.28}O₂ (50%) + | No additive | 822 mAh | 30 mΩ | 31 mΩ |
| Ex. 14 | Li_{1.06}Mn_{1.89}Mg_{0.05}O₄ (50%) | | | | |
| Comp. | Li_{1.07}Ni_{0.56}Mn_{0.37}O₂ (50%) + | No additive | 895 mAh | 39 mΩ | 39 mΩ |
| Ex. 15 | Li_{1.06}Mn_{1.89}Mg_{0.05}O₄ (50%) | | | | |

The results shown in Table 1 demonstrate that the non-aqueous electrolyte secondary batteries A1 to A6 of Examples according to the present invention, each of which employed the lithium-containing transition metal composite oxide and the electrolyte solution containing an oxalato complex salt (LiBOB), exhibited lower I-V resistances than Battery B1 of Comparative Example 1, which did not contain the oxalato complex salt, on both the charge side and the discharge side. Examples 1, 3, and 4, in which the concentration of LiBOB in the electrolyte solution is within the range of from 0.1M to 0.2M, exhibited particularly low I-V resistances on both the discharge side and the charge side.

From a comparison between Battery B2 of Comparative Example 2 and Battery B3 of Comparative Example 3, it is demonstrated that the effect of improving the input/output power characteristics cannot be obtained when using a lithium-containing transition metal composite oxide that is outside range of the present invention, even if an oxalato complex salt is added to the electrolyte solution. Therefore, it is understood that the effect of improving the input/output power characteristics resulting from the addition of an oxalato complex salt is inherent to the case of using the lithium-containing transition metal composite oxide according to the invention.

From a comparison between Battery A6 of Example 6 and Battery A1 of Example 1, it is apparent that Battery A6, which employs the positive electrode active material in which a titanium-containing oxide is adhered to the particle surfaces of the lithium-containing transition metal composite oxide, exhibits even lower I-V resistances than Battery Al on both the charge side and the discharge side.

From a comparison between Battery A6 of Example 6 and Battery B4 of Comparative Example 4, it is apparent that the use of the electrolyte solution containing an oxalato complex salt can reduce the charge side and discharge side I-V resistances also when using the positive electrode active material in which a titanium-containing oxide is adhered to the particle surfaces of the lithium-containing transition metal composite oxide.

More specifically, from a comparison between Battery Al of Example 1 and Battery B1 of Comparative Example 1, from a comparison between Battery A7 of Example 7 and Battery B5 of Comparative Example 5, and from a comparison between Battery A8 of Example 8 and Battery B6 of Comparative Example 6, it is apparent that the I-V resistance on both the discharge side and the charge side can be reduced when using the lithium-containing transition metal composite oxide according to the present invention and also adding an oxalato complex salt to the electrolyte solution. It is also demonstrated that the use of the electrolyte solution containing an oxalato complex salt provides a more significant effect of reducing the I-V resistance especially when c is 0 in the foregoing general formula. Furthermore, it is also demonstrated that the use of the electrolyte solution containing an oxalato complex salt provides an even more significant effect of reducing the I-V resistance when c is 0 and also a=b in the foregoing general formula.

On the other hand, from a comparison between Battery B7 of Comparative Example 7 and Battery B8 of Comparative Example 8, it is apparent that, when using the lithium-containing transition metal composite oxide that is outside the range of the present invention, even the use of the electrolyte solution containing an oxalato complex salt cannot provide the effect of reducing the I-V resistance.

Moreover, from a comparison between Battery A9 of Example 9 and Battery B 11 of Comparative Example 11, from a comparison between Battery A10 of Example 10 and Battery B12 of Comparative Example 12, and from a comparison between Battery A11 of Example 11 and Battery B15 of Comparative Example 15, it is apparent that the use of the electrolyte solution containing an oxalato complex salt provides a more significant effect of reducing the I-V resistance even when using the positive electrode active material containing the lithium-containing transition metal composite oxide according to the present invention together with the lithium-manganese composite oxide having a spinel structure.

Moreover, from a comparison between Battery A7 of Example 7 and Battery A11 of Example 11 and from a comparison between Battery A8 of Example 8 and Batteries A9 and A10 of Examples 9 and 10, it is apparent that the use of a positive electrode active material containing the lithium-containing transition metal composite oxide according to the present invention together with the lithium-manganese composite oxide having a spinel structure provides a more significant effect of reducing the I-V resistance than in the case that the lithium-containing transition metal composite oxide according to the present invention was used alone as the positive electrode active material.

On the other hand, from a comparison between Battery B13 of Comparative Example 13 and Battery B14 of Comparative Example 14, it is apparent that, when using the lithium-containing transition metal composite oxide that is outside the range of the present invention together with the lithium-manganese composite oxide having a spinel structure, even the use of the electrolyte solution containing an oxalato complex salt cannot provide the effect of reducing the I-V resistance.

Furthermore, from a comparison between Battery B9 of Comparative Example 9 and Battery B10 of Comparative Example 10, it is apparent that the effect of reducing the I-V resistance cannot be obtained when the lithium-manganese composite oxide having a spinel structure alone is used as the positive electrode active material, even if the electrolyte solution containing an oxalato complex salt is used.

As described above, the present invention makes it possible to reduce both the charge-side and discharge-side I-V resistances at room temperature and to improve the input/output power characteristics.

### REFERENCE EXPERIMENT 1

### Preparation of Positive Electrode

Li_{1.06}Ni_{0.47}Mn_{0.47}O₂ was used as the lithium-containing transition metal oxide, and a slurry was prepared in the same manner as described in Example 1. The resultant slurry was applied onto an aluminum foil, then dried, and pressure-rolled. Thereafter, the resultant article was cut into a predetermined size. Then, an aluminum current collector tab was attached thereto. Thus, a positive electrode of Reference Experiment 1 was fabricated.

### Preparation of Wound Electrode Assembly

A wound electrode assembly was prepared by winding the positive electrode prepared in the just-described manner and a negative electrode, with a polyethylene separator interposed therebetween.

### Preparation of Non-Aqueous Electrolyte

A non-aqueous electrolyte was prepared in the following manner. LiPF₆ as a solute was dissolved at a concentration of 1 mole/liter in a solvent of 3:3:4 volume ratio mixture of ethylene carbonate (EC), methyl ethyl carbonate (MEC), and dimethyl carbonate (DMC), and 1 weight % of vinylene carbonate (VC) was dissolved therein.

### Fabrication of Non-Aqueous Electrolyte Secondary Battery

A three-electrode cell was prepared using the positive electrode prepared in the above-described manner as the working electrode, the negative electrode as the counter electrode, and metallic lithium as the reference electrode. The above-described non-aqueous electrolyte was filled in the three-electrode cell, whereby a non-aqueous electrolyte secondary battery X1 of Reference Experiment 1 was prepared.

### Evaluation of Reactivity Between Charged Positive Electrode and Electrolyte Solution

Specifically, the prepared non-aqueous electrolyte secondary battery X1 was charged at a constant current density of 0.2 mA/cm² to 4.3 V (vs. Li/Li⁺) at 25° C., and discharged at a constant voltage of 4.3 V (vs. Li/Li⁺) at 25° C. Thereafter, 5 mg of the lithium-containing transition metal oxide that was peeled from the electrode plate and 3 mg of the electrolyte solution were enclosed in an A1 container, and the reactivity between the electrolyte solution and the positive electrode active material was evaluated by a DSC measurement.

### REFERENCE EXPERIMENT 2

A DSC measurement was conducted in the same manner as described in Reference Experiment 1, except that the lithium-containing transition metal oxide was Li_{1.06}Ni_{0.52}Mn_{0.42}O₂.

### REFERENCE EXPERIMENT 3

A DSC measurement was conducted in the same manner as described in Reference Experiment 1, except that the lithium-containing transition metal oxide was Li_{1.06}Ni_{0.56}Mn_{0.38}O₂.

### REFERENCE EXPERIMENT 4

A DSC measurement was conducted in the same manner as described in Reference Experiment 1, except that the lithium-containing transition metal oxide was Li_{1.06}Ni_{0.66}Mn_{0.28}O₂.

The results of the reference experiments are shown in Table 2 below.

TABLE 2

| | | | Exothermic peak |
|---|---|---|---|
| | Composition | Ratio a/b | temperature |
| Reference | Li_{1.06}Ni_{0.47}Mn_{0.47}O₂ | 1.0 | 305° C. |
| Experiment 1 | | | |
| Reference | Li_{1.06}Ni_{0.52}Mn_{0.42}O₂ | 1.2 | 298° C. |
| Experiment 2 | | | |
| Reference | Li_{1.06}Ni_{0.56}Mn_{0.38}O₂ | 1.5 | 296° C. |
| Experiment 3 | | | |
| Reference | Li_{1.06}Ni_{0.66}Mn_{0.28}O₂ | 2.3 | 224° C. |
| Experiment 4 | | | |

As clearly seen from the results shown in Table 2, it was found that the exothermic peak temperature reduces considerably and the thermal stability deteriorates significantly in the case of Reference Experiment 4, in which the ratio a/b in the lithium-containing transition metal oxide is a/b>2.0, in comparison with the cases in which a/b≦2.0 (Reference Experiments 1 to 3). Therefore, it is preferable that the ratio a/b in the lithium-containing transition metal oxide according to the present invention be a/b≦2.0, from the viewpoint of thermal stability.

Only selected embodiments have been chosen to illustrate the present invention. To those skilled in the art, however, it will be apparent from the foregoing disclosure that various changes and modifications can be made herein without departing from the scope of the invention as defined in the appended claims. Furthermore, the foregoing description of the embodiments according to the present invention is provided for illustration only, and is not intended to limit the invention as defined by the appended claims and their equivalents.

What is claimed is:

1. A non-aqueous electrolyte secondary battery comprising:
a positive electrode containing a positive electrode active material, the positive electrode active material comprising a lithium-containing transition metal oxide having a layered structure and being represented by the general formula Li₁₊ₓ(NiₐMn_{b}Co_{c})O_{2+α}, where x+a+b+c=1, 0.7≦a+b, 0<x≦0.1, 0≦c/(a+b)<0.35, 0.7≦a/b≦2.0, and −0.1≦α≦0.1;
a negative electrode containing a negative electrode active material; and
a non-aqueous electrolyte having lithium ion conductivity and containing a lithium salt having an oxalato complex as an anion.

2. A non-aqueous electrolyte secondary battery comprising:
a positive electrode containing a positive electrode active material, the positive electrode active material comprising a lithium-containing transition metal oxide in which a titanium-containing oxide is adhered to a surface thereof, the lithium-containing transition metal oxide having a layered structure and being represented by the general formula Li₁₊ₓ(NiₐMn_{b}Co_{c})O_{2+α}, where x+a+b+c=1, 0.7≦a+b, 0<x≦0.1, 0≦c/(a+b)<0.35, 0.7≦a/b≦2.0,and −0.1≦α≦0.1;
a negative electrode containing a negative electrode active material; and
a non-aqueous electrolyte having lithium ion conductivity and containing a lithium salt having an oxalato complex as an anion.

3. The non-aqueous electrolyte secondary battery according to claim 1, wherein the positive electrode active material further comprises a lithium-manganese composite oxide having a spinel structure.

4. The non-aqueous electrolyte secondary battery according to claim 2, wherein the positive electrode active material further comprises a lithium-manganese composite oxide having a spinel structure.

5. The non-aqueous electrolyte secondary battery according to claim 1, wherein c is 0.

6. The non-aqueous electrolyte secondary battery according to claim 2, wherein c is 0.

7. The non-aqueous electrolyte secondary battery according to claim 3, wherein c is 0.

8. The non-aqueous electrolyte secondary battery according to claim 4, wherein c is 0.

9. The non-aqueous electrolyte for secondary batteries according to claim 1, wherein the lithium salt having an oxalato complex as an anion is lithium bis(oxalato)borate.

10. The non-aqueous electrolyte for secondary batteries according to claim 2, wherein the lithium salt having an oxalato complex as an anion is lithium bis(oxalato)borate.

11. The non-aqueous electrolyte for secondary batteries according to claim 1, wherein the lithium salt having an oxalato complex as an anion is contained in the non-aqueous electrolyte at a concentration of from 0.05 mole/liter to 0.3 mole/liter.

12. The non-aqueous electrolyte for secondary batteries according to claim 2, wherein the lithium salt having an oxalato complex as an anion is contained in the non-aqueous electrolyte at a concentration of from 0.05 mole/liter to 0.3 mole/liter.

13. The non-aqueous electrolyte for secondary batteries according to claim 9, wherein the lithium salt having an oxalato complex as an anion is contained in the non-aqueous electrolyte at a concentration of from 0.05 mole/liter to 0.3 mole/liter.

14. The non-aqueous electrolyte for secondary batteries according to claim 10, wherein the lithium salt having an oxalato complex as an anion is contained in the non-aqueous electrolyte at a concentration of from 0.05 mole/liter to 0.3 mole/liter.

15. The non-aqueous electrolyte secondary battery according to claim 1, wherein the negative electrode active material is an amorphous carbon-coated graphite.

16. The non-aqueous electrolyte secondary battery according to claim 2, wherein the negative electrode active material is an amorphous carbon-coated graphite.

17. The non-aqueous electrolyte secondary battery according to claim 1, wherein the non-aqueous electrolyte contains vinylene carbonate.

18. The non-aqueous electrolyte secondary battery according to claim 2, wherein the non-aqueous electrolyte contains vinylene carbonate.
